(19) United States
(12) Patent Application Publication
(10) Pub. No.: US 2020/0298545 A1
(43) Pub. Date: Sep. 24, 2020

(54) APPARATUS AND METHOD OF MANUFACTURING AN ELASTIC COMPOSITE STRUCTURE FOR AN ABSORBENT SANITARY PRODUCT

(71) Applicant: Curt G. Joa, Inc., Sheboygan Falls, WI (US)

(72) Inventors: Robert E. Andrews, Falmouth, ME (US); David E. Schuette, Kiel, WI (US); Jeffrey W. Fritz, Plymouth, WI (US)

(21) Appl. No.: 16/946,106

(22) Filed: Jun. 5, 2020

Related U.S. Application Data
(63) Continuation-in-part of application No. 16/721,414, filed on Dec. 19, 2019.
(60) Provisional application No. 62/789,058, filed on Jan. 7, 2019.

(51) Int. Cl. B32B 37/00; A61F 13/474; A61F 13/49; A61F 13/15; B32B 7/10; B32B 5/26; B32B 3/26; B32B 7/05; B32B 27/08; B32B 5/18; B32B 5/32

(52) U.S. Cl. CPC B32B 37/0046; B32B 2037/0092; A61F 13/4902; A61F 13/15699; B32B 7/10; B32B 5/26; B32B 3/26; B32B 7/05; B32B 27/08; B32B 5/18; B32B 5/32; B32B 37/0076; A61F 2013/49025; B32B 2555/02; B32B 2262/02; A61F 13/474

(57) ABSTRACT

An apparatus and method for manufacturing an elastic composite structure for an absorbent sanitary product includes a rotary anvil comprising a face surface, a plurality of non-linear ridges defined by a first plurality of grooves in the face surface, and a plurality of projections in each of the plurality of non-linear ridges. Each projection of the plurality of projections comprises a contact surface having parallel facing surfaces and parallel end surfaces.

### CROSS-REFERENCE TO RELATED APPLICATIONS

The present invention is a continuation-in-part of and claims priority to U.S. Provisional patent application Ser. No. 16/721,414 filed Dec. 19, 2019, which claims priority to U.S. Provisional Patent Application Ser. No. 62/789,058 filed Jan. 7, 2019, the disclosure of which is incorporated herein by reference in its entirety.

### BACKGROUND OF THE INVENTION

Embodiments of the invention relate generally to absorbent sanitary products and, more particularly, to an improved apparatus and method for manufacturing an elastic composite structure for use in an absorbent sanitary product that minimizes or eliminates the use of consumable adhesives such as glue.

Absorbent sanitary products, such as disposable diapers, are typically equipped with elastic composite structures that include one or more elastic threads. These elastic composite structure s are positioned at various locations throughout the product, including in the waistbands, leg cuff regions, and throughout all or portions of the front or back panels of the product. During the typical manufacturing process of an elastic composite structure, the elastic threads are held in a tensioned state and an adhesive is used to secure the elastic threads between the two facing layers of non-woven materials or webs. The tension in the elastic threads is subsequently released, causing the web material to pucker or fold in the areas that contain the adhered elastic threads.

The use of adhesives to bond the elastic threads within elastic composite structures presents a number of disadvantages in both the end product and manufacturing method, including costs associated with the consumable material and undesirable tactile properties of the end product (e.g., stiffness). While thermal or ultrasonic welding techniques have been proposed as alternatives for bonding elastic threads within an elastic composite structure, movement or shifting of the elastic threads between or outside of notches on the anvil during the manufacturing process may result in a given elastic thread breaking or being unanchored over one or more portions of its length.

Accordingly, there is a need for an improved apparatus and method for fabricating an elastic composite structure of an absorbent sanitary product that reduces thread breakage and improves the reliability of bonds that anchor elastic threads in position within an elastic composite structure. It would further be desirable for such an apparatus and method to eliminate or minimize the use of consumable adhesives to secure the elastic threads to the facing web layers.

### BRIEF STATEMENT OF THE INVENTION

In accordance with one aspect of the invention, a rotary anvil comprises a face surface, a plurality of non-linear ridges defined by a first plurality of grooves in the face surface, and a plurality of projections in each of the plurality of non-linear ridges. Each projection of the plurality of projections comprises a contact surface having parallel facing surfaces and parallel end surfaces.

In accordance with another aspect of the invention, a method of manufacturing a rotary anvil comprises providing a rotary anvil having a face surface, removing material from the face surface to form a plurality of non-linear welding lines in the rotary anvil, and forming a plurality of projections in the plurality of non-linear welding lines. Forming the plurality of projections comprises creating a contact surface for each projection of the plurality of projections, the contact surface having parallel facing surfaces and parallel end surfaces. The parallel facing surfaces of each contact surface are parallel to one another, and the parallel end surfaces of each contact surface are parallel to one another.

In accordance with another aspect of the invention, an elastic composite structure comprises a first web layer, a second web layer coupled to the first web layer by a non-linear bond pattern comprising at least non-linear one bond line having at least one pair of adjacent bonds, and at least one elastic thread extending through a passage defined by facing edges of the at least one pair of adjacent bonds. Each bond in the at least one bond line comprises parallel facing surfaces and parallel end surfaces orthogonal to the parallel facing surfaces.

These and other advantages and features will be more readily understood from the following detailed description of preferred embodiments of the invention that is provided in connection with the accompanying drawings.

### BRIEF DESCRIPTION OF THE DRAWINGS

The drawings illustrate embodiments presently contemplated for carrying out the invention.

In the drawings:

FIG. 1 is a schematic perspective view of a portion of a manufacturing line for fabricating an elastic composite structure.

FIG. 2 is a schematic perspective view of a portion of the manufacturing line illustrated in FIG. 1.

FIG. 3 is a cross-sectional view of a portion of a bonding unit usable with the manufacturing line of FIG. 1, according to one embodiment of the invention.

FIG. 4A is a cross-sectional view of a portion of an elastic composite structure fabricated using the bonding unit of FIG. 3 in its relaxed or non-tensioned state, according to one embodiment of the invention.

FIG. 4B is a cross-sectional view of a portion of an elastic composite structure fabricated using the bonding unit of FIG. 3 in its relaxed or non-tensioned state, according to another embodiment of the invention.

FIG. 5 is a cross-sectional view of an exemplary elastic strand of the elastic composite structure of FIG. 4 in its relaxed or non-tensioned state.

FIG. 6 is a cross-sectional view of a portion of a bonding unit usable with the manufacturing line of FIG. 1, according to another embodiment of the invention.

FIG. 7 is a cross-sectional view of a portion of a bonding unit usable with the manufacturing line of FIG. 1, according to another embodiment of the invention.

FIG. 8 is a cross-sectional view of a portion of a bonding unit usable with the manufacturing line of FIG. 1, according to another embodiment of the invention.

FIG. 9 is a cross-sectional view of a portion of a bonding unit usable with the manufacturing line of FIG. 1, according to another embodiment of the invention.

FIG. 10 is a cross-sectional view of a portion of a bonding unit usable with the manufacturing line of FIG. 1, according to another embodiment of the invention.

FIG. 11A is a cross-sectional view of a portion of a bonding unit usable with the manufacturing line of FIG. 1, according to another embodiment of the invention.

FIG. 11B is a cross-sectional view of a portion of an elastic composite structure fabricated using the bonding unit of FIG. 11A in its relaxed or non-tensioned state.

FIG. 12 is a front view of a rotary anvil usable with the manufacturing line of FIG. 1, according to an embodiment of the invention.

FIG. 12A is a detailed view of a portion of the rotary anvil of FIG. 12.

FIG. 13 is a top view of a portion of an elastic composite structure shown in its elongated or tensioned state, according to an embodiment of the invention.

FIG. 13A is a detailed view of a portion of the elastic composite structure of FIG. 13 shown in its elongated or tensioned state.

FIG. 14 is a cross-sectional view of a multifilament elastic thread usable to manufacture the elastic composite structure of FIG. 13.

FIG. 15 is a front view of a rotary anvil usable with the manufacturing line of FIG. 1, according to another embodiment of the invention.

FIG. 15A is a front view of a rotary anvil usable with the manufacturing line of FIG. 1, according to another embodiment of the invention.

FIG. 16 is a front view of a rotary anvil usable with the manufacturing line of FIG. 1, according to another embodiment of the invention.

FIG. 16A is a front view of a rotary anvil usable with the manufacturing line of FIG. 1, according to another embodiment of the invention.

FIGS. 17-21 illustrate cross-sectional views taken along line 17-21 of FIGS. 15 and 16 showing steps for manufacturing the rotary anvils of FIGS. 15 and 16 according to an embodiment of the invention.

FIGS. 22-23 illustrate steps for manufacturing the rotary anvils of FIGS. 15 and 16 according to another embodiment of the invention.

FIG. 24 illustrates a rotary anvil manufactured using the step of FIG. 23 according to another embodiment of the invention.

FIGS. 25A-28A illustrate cross-sectional views taken along line 25-28 of FIGS. 15 and 16 showing steps for manufacturing the rotary anvils of FIGS. 15 and 16 according to another embodiment of the invention.

FIGS. 25B-28B illustrate top views corresponding to the steps shown in FIGS. 25A-28A.

FIGS. 29A-33A and 29B-33B illustrate cross-sectional and top views of rotary anvils manufactured according to alternative embodiments of the invention.

FIGS. 34-36 illustrate orthogonal views of alternative electrodes according to an embodiment of the invention.

### DETAILED DESCRIPTION

Embodiments of the present invention provide for a method and apparatus for manufacturing an elastic composite structure usable in an absorbent sanitary product such as, for example, a diaper, disposable adult pant, or feminine care product.

During the manufacture of absorbent sanitary products, it is often desirable to secure elastic threads between facing layers of non-woven material to form contoured or elasticized regions within the product. Such products are typically manufactured on an assembly or manufacturing line in which the product moves substantially continually longitudinally in what is referred to as the “machine direction.”

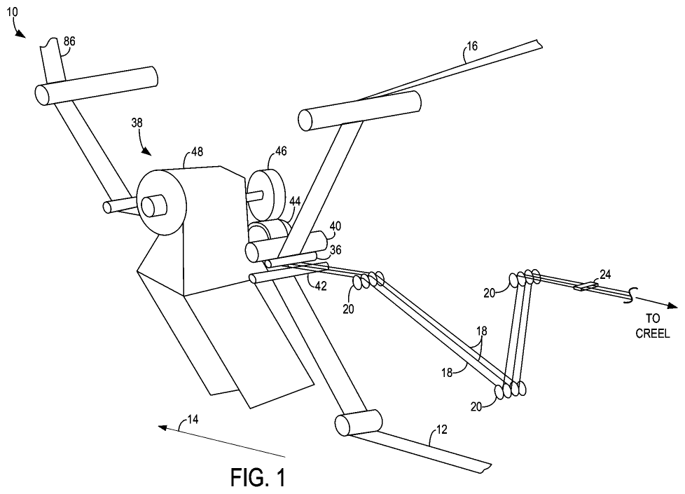

Referring now to FIG. 1, a portion of an exemplary manufacturing line 10 is illustrated according to one embodiment of the invention. As shown, a first web layer 12 is fed in the machine direction 14. A second web layer 16 is similarly fed in the machine direction 14. First web layer 12 and second web layer 16 are materials capable of fusing to one another upon application of an applied energy that causes one or both of the webs 12, 16 to soften or melt and join together. First and second web layers 12, 16 may be the same type of material or different materials according to alternative embodiments. As non-limiting examples, first and second web layers 12, 16 may include nonwoven materials, woven materials, films, foams, and/or composites or laminates of any of these material types.

A series of individual elastic threads 18 are positioned between the first and second web layers 12, 16. The elastic threads 18 travel in the machine direction 14 under tension from a creel assembly (not shown) or similar device. The elastic threads 18 may be composed of any suitable elastic material including, for example, sheets, strands or ribbons of thermoplastic elastomers, natural or synthetic rubber, or LYCRA, as non-limiting examples. Each elastic thread 18 may be provided in the form of an individual elastomeric strand or be a manufactured multifilament product that includes many individual elastomeric filaments joined together, such as by a dry-spinning manufacturing process, to form a single, coalesced elastic thread 18. Each elastic thread 18 may be in the range of approximately 200-1500 decitex (dTex), in non-limiting embodiments. In an embodiment where an elastic thread 18 is a multifilament product, the elastic thread 18 may have an overall decitex of 400 dTex, in an exemplary and non-limiting embodiment, with the individual elastomeric filaments of the elastic thread 18 individually having a decitex of ten percent or less of the overall 400 dTex value. As just a few examples, a multifilament thread with a decitex of 680 and up may include 55 individual elastomeric filaments while a multifilament thread with a decitex lower than 680 may include 47 individual elastomeric filaments.

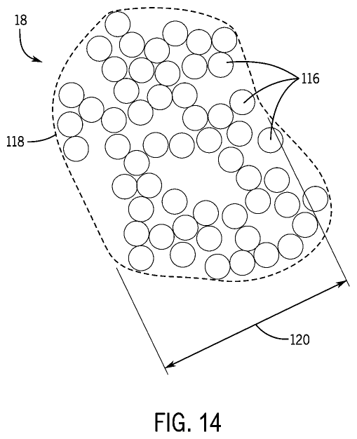

Elastic threads 18 may have any suitable cross-sectional shape that facilitates formation of an elastic composite structure having desired elasticity, visual aesthetic, and manufacturability. As non-limiting examples, elastic threads 18 may have a cross-sectional shape that is round, rectangular, square, or irregular as may be the case where each elastic thread 18 is a multifilament product (as illustrated in detail in FIG. 14).

While first web layer 12 and second web layer 16 are depicted in FIG. 1 and described herein as physically separate components, it is contemplated that alternative embodiments may utilize a unitary web structure that is folded to capture the elastic threads 18 between upper and lower layers of the unitary web structure. In such an embodiment, the portion of the unitary structure positioned below the elastic threads 18 would be referred to as the first web layer 12 and the portion of the unitary structure positioned above the elastic threads 18 would be referred to as the second web layer 16.

Manufacturing line 10 includes one or more guide rollers 20 that are employed to transport, accurately position and (optionally) tension the elastic threads 18 as it travels in the machine direction 14. In some embodiments, manufacturing line 10 may include one or more optional tension monitoring devices 24 (shown in phantom) that are positioned along the path of travel of the elastic threads 18. In such an embodiment, feedback from the tension monitoring devices 24 may be utilized to control the tension (i.e., elongation) in the elastic threads 18 as they travel in the machine direction 14.

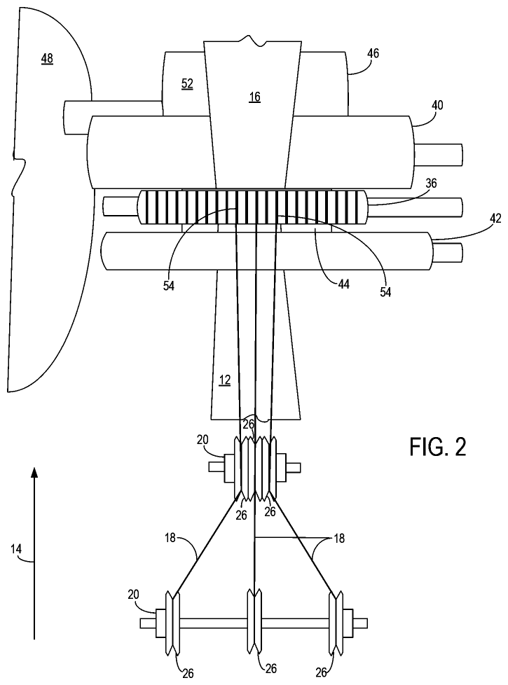

As shown in further detail in FIG. 2, each respective elastic thread 18 is positioned within a respective guiding section 26 of guide rollers 20. Doing so maintains separation between the adjacent elastic threads 18. In the illustrated embodiment, guiding section 26 includes notches that aid in alignment and guiding of the elastic threads 18. Notches may be v-shaped as shown, have curved or other alternative geometries, or be omitted entirely in alternative embodiments.

Guide rollers 20 operate to accurately position and tension individual elastic threads 18 as they travel toward a strand guide roller 36 that is positioned upstream of bonding unit 38, which is referred to hereafter as ultrasonic bonding apparatus 38. Manufacturing line 10 also includes one or more structures that are configured to transport and guide the first and second web layers 12, 16 in the machine direction 14. In the illustrated embodiment, these guide structures include an upper roller 40 and a lower roller 42 are positioned to guide the first web layer 12 and the second web layer 16, respectively, toward the ultrasonic bonding apparatus 38.

Ultrasonic bonding apparatus 38 may be a rotary ultrasonic welding system or a blade ultrasonic welding system in alternative embodiments. In the illustrated embodiment, ultrasonic bonding apparatus 38 is a rotary ultrasonic welding system that includes a rotary anvil 44 and a horn 46 that cooperate with each other to bond the first web layer 12 to the second web layer 16. The elastic threads 18 are secured or anchored in position relative to the first and second web layers 12, 16 as described in detail below. Ultrasonic bonding apparatus 38 also includes one or more frames 48 that support and/or house a motor (not shown) that drives the horn 46, a vibration control unit (not shown) that causes the horn 46 to vibrate, and a second motor (not shown) that drives the anvil 44. The horn 46 and anvil 44 are positioned in a spaced relationship relative to one another to facilitate ultrasonically bonding the first and second web layers 12, 16 to one another while the elastic threads 18 are held in tension in the space between the horn 46 and anvil 44. While horn 46 is illustrated as a rotary horn in FIG. 1, a stationary horn may be used in alternative embodiments.

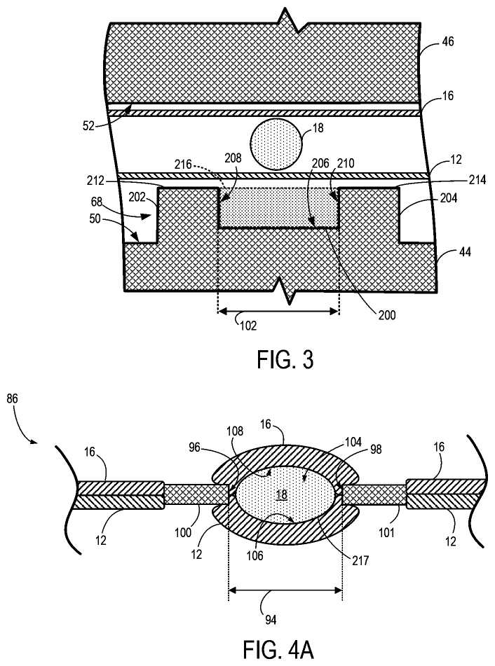

The face 50 of the anvil 44 includes an arrangement of projections and notches that facilitate securing the combined elastic thread assemblies 34 in position relative to the first and second web layers 12, 16. Exemplary embodiments of this arrangement of projections and notches are described in detail below relative to FIGS. 3-11. In one non-limiting embodiment, the face 52 of the horn 46 has a smooth or substantially smooth surface contour. In alternative embodiments, face 52 may include an arrangement of projections and/or notches that mate or align with the surface pattern of the anvil 44 to further facilitate bonding the first and second web layers 12, 16 together and securing the elastic threads 18 in position relative to the first and second web layers 12, 16.

While embodiments of the invention are described relative to an ultrasonic bonding assembly and ultrasonic bonding technique, it is contemplated that the techniques described herein may be extended to any other known thermal or pressure bonding techniques.

FIG. 2 is a view of a portion of the manufacturing line 10 upstream of the ultrasonic bonding apparatus 38 looking into the machine direction 14. As shown, the elastic threads 18 are fed outward from respective guiding sections 26 in the guide rollers 20 and toward strand guide roller 36. In the embodiment, strand guide roller 36 includes an array of notches 54 that aid in aligning and guiding the elastic threads as they are received between the horn 46 and anvil 44. These notches 54 may be evenly spaced across all of the strand guide roller 36 in the manner shown or may span only a portion thereof in an alternative embodiment. In yet other embodiments, the notches 54 may be positioned at uneven intervals along the length of strand guide roller 36 depending upon design specifications and the desired placement and spacing of the elastic threads 18 in the resulting elastic composite structure.

Referring now to FIG. 3, a cross-sectional view of a portion of the anvil 44 and horn 46 is provided according to one embodiment of the invention. As shown, the face 50 of the anvil 44 includes a welding line 68 that is defined by at least one notch 200, which is positioned between a corresponding pair of projections 202, 204. While only one instance of a notch 200 and corresponding pair of projections 202, 204 is illustrated in FIG. 3, it is contemplated that each welding line 68 on the anvil 44 may alternatively include multiple notches 200, with each notch 200 similarly arranged between a corresponding pair of projections 202, 204. In the embodiment shown, notch 200 has a u-shaped geometry defined by a bottom surface 206 and facing surfaces 208, 210 of the projections 202, 204. One or more of surfaces 206, 208, 210 may be planar, as shown, or curved in alternative embodiments.

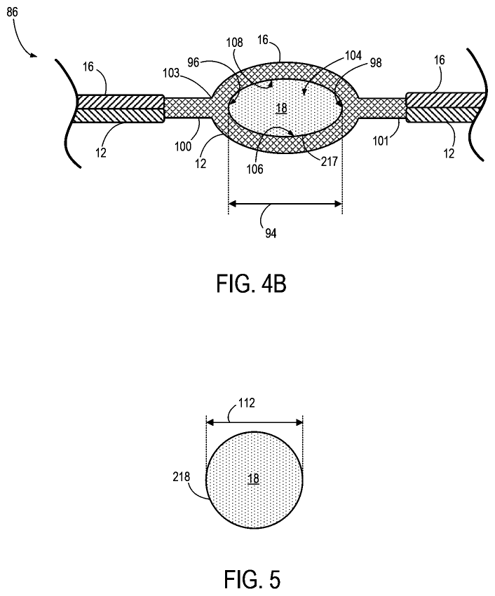

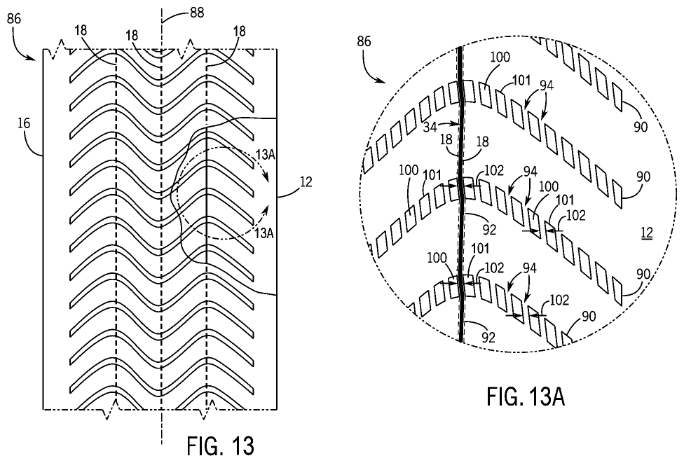

During the manufacturing process, the first and second web layers 12, 16 are positioned between the face 50 of the anvil 44 and the face 52 of the horn 46. An elastic thread 18 is positioned between the first and second web layers 12, 16 in a tensioned state and aligned above notch 200. As shown in FIGS. 4A and 4B and with continued reference to FIG. 3, the first and second web layers 12, 16 are bonded together by a pair of bonds 100, 101 at locations corresponding to the land surfaces 212, 214 of the respective projections 202, 204. Thus bonds 100, 101 each have a width that corresponds to the width of land surfaces 212, 214. Depending on the operating parameters of the ultrasonic bonding apparatus 38 and/or the geometry and configuration of the notches and projections on the anvil and/or horn, the resulting pair of adjacent bonds 100, 101 either may be discrete, discontinuous bonds 100, 101 as shown in FIG. 4A, or part of a continuous fusion bond 103 that fuses the facing web layers 12, 15 together at bond points 100, 101 and fuses one or both of the facing web layers 12, 16 to the elastic thread 18, as shown in FIG. 4B. The bonding operation creates a manufactured elastic composite structure 86 as shown in FIG. 13.

When the manufactured elastic composite structure 86 shown in FIG. 13 is permitted to relax, each elastic thread 18 will attempt to swell or expand to return to its non-tensioned or relaxed state within passage 104. Passage 104 has a cross-sectional area 217 that is dictated by the cross-sectional area 216 of the notch 200 on anvil 44. Thus, the cross-sectional area 217 of passage 104 is equal to or substantially equal to the cross-sectional area 216 of the notch 200. Notch 200 is sized to have a cross-sectional area 216 that is less than the cross-sectional area 218 of the elastic thread 18 in its non-tensioned or relaxed state, which is illustrated in FIG. 5. As the elastic thread 18 expands, it becomes anchored or trapped in the passage 104 formed between the upward facing surface 106 of the first web layer 12, the downward facing surface 108 of the second web layer 16, and the facing edges 96, 98 of a pair of adjacent bonds 100, 101.

As shown in FIG. 4, the elastic thread 18 deforms as it expands due to the relatively shallow geometry of the notch 200. Depending on the shape and dimensions of notch 200 and the cross-sectional area 218 of the non-tensioned elastic thread 18, the elastic thread 18 may expand to completely fill the passage 104, as shown in FIG. 4. Alternatively, the elastic thread 18 may expand to a position where the elastic thread 18 fills only a portion of the passage 104. In such an embodiment, the portion of the elastic thread 18 adjacent bonds 100, 101 would be secured in position relative to web layers 12, 16 by virtue of contact between the elastic thread 18 and facing surfaces 106, 108 of the web layers 12, 16 with a gap formed between the elastic thread 18 and one or both of the facing edges 96, 98 of adjacent bonds 100, 101.

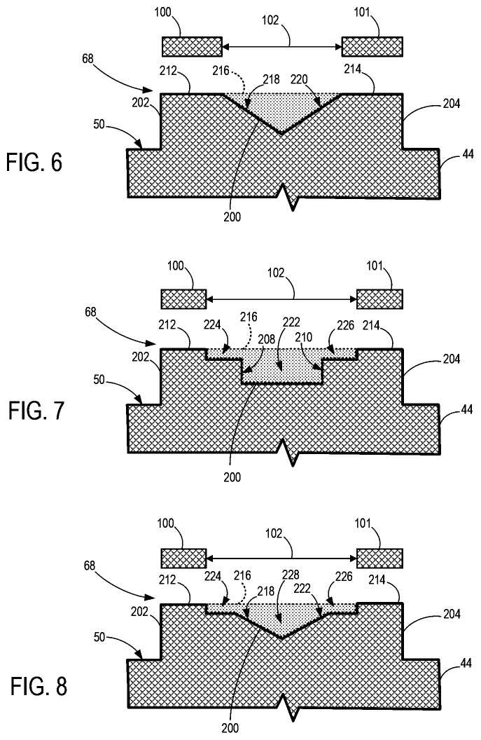

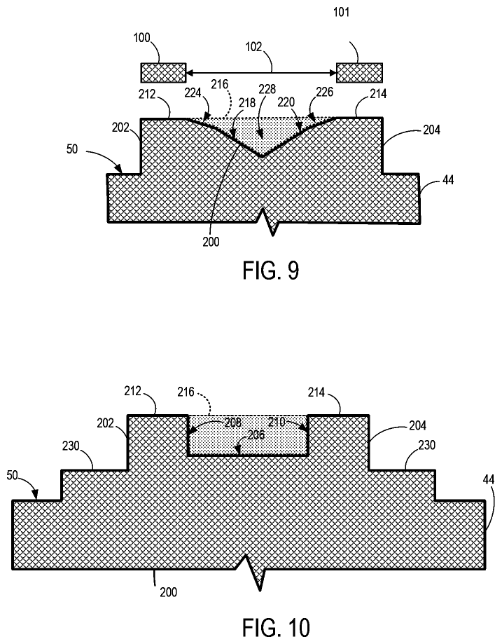

FIGS. 6, 7, 8, and 9 depict notch configurations according to alternative embodiments of the invention. A cross-sectional view of the resulting pair of adjacent bonds 100, 101 between the first and second web layers 12, 16 is provided above the land surfaces 212, 214 of the respective projections 202, 204 for ease of reference. Other portions of the elastic composite structure 86 are omitted for clarity purposes. In FIG. 6, notch 200 has a v-shaped geometry formed by opposing angled surfaces 218, 220. The notches 200 in FIGS. 7 and 8 have stepped configurations. In FIG. 7, notch 200 includes a u-shaped central region 222 defined by bottom surface 206 and two facing surfaces 208, 210 and two opposing side regions 224, 226. The notch 200 of FIG. 8 includes similarly configured side regions 224, 226 with a v-shaped central region 228 defined by opposing angled surfaces 218, 220. FIG. 9 depicts a modified stepped geometry where the angled surfaces 218, 220 of notch 200 have a different slope in the central region 228 of the notch 200 than in the opposing side regions 224, 226. The surfaces that define the notches 200 in FIGS. 6-9 may be straight, as shown, curved, or some mixture of curved and straight in alternative embodiments.

Each of notches 200 in FIGS. 6-9 has a cross sectional area 216 that is smaller than the cross-sectional area of the elastic thread 18 in its non-tensioned state. The notches 200 of FIGS. 6-9 define a resulting pair of adjacent bonds 100, 101 that are spaced apart by a gap or distance 102 that is greater than the strand diameter 112 of the elastic thread 18 when in its non-tensioned state.

As used herein the phrase “strand diameter” refers to the smallest measurable cross-sectional width of the elastic thread 18 in its non-tensioned state. In embodiments where a given elastic thread 18 is a monofilament structure, the strand diameter is the minor diameter or smallest measurable width of the monofilament structure in its non-tensioned state. In embodiments where a given elastic thread 18 is a structure that includes many individual filaments 116 (i.e., elastic thread 18 is a multi-filament structure), the elastic thread 18 typically will have an irregular cross-sectional area similar to that shown in FIG. 14. The strand diameter of such a multifilament structure is to be understood as the smallest distance 120 between opposite edges of an outline that generally defines the irregular cross-sectional area. The cross-sectional area of the multifilament structure may be measured as the cross-sectional area within a perimeter 118 drawn to surround all of the individual filaments 116 or calculated as the summed total of the cross-sectional area of each of the individual filaments 116.

FIG. 10 depicts a portion of anvil 44 according to yet another embodiment of the invention. In this embodiment, notch 200 and the pair of flanking projections 202, 204 are formed atop a step 230 that is elevated above the face 50 of the anvil 44. While only one notch 200 and corresponding pair of projections 202, 204 is illustrated atop step 230, alternative embodiments may include any number of notches 200 and corresponding projections 202, 204. Notch 200 may have the u-shaped geometry shown in FIG. 3 or any of the alternative notch geometries illustrated in FIGS. 6-9 or otherwise described herein.

Each of FIG. 3, FIG. 6, FIG. 7, FIG. 8, FIG. 9, and FIG. 10 is to be understood as illustrating one exemplary and non-limiting configuration of notch 200. In alternative embodiments, anvil 44 may include one or more notches 200 that has any shape or surface topology, including straight surfaces, curved surfaces, or any combination thereof that results in a notch 200 having a cross-sectional area 216 that is smaller than the cross-sectional area 218 of the corresponding elastic thread 18 when in its non-tensioned state.

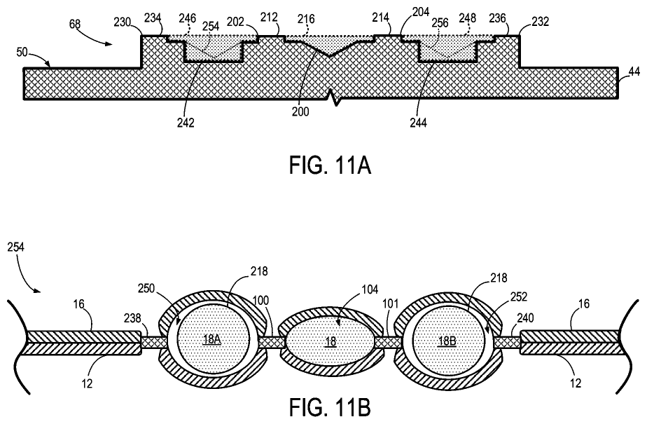

FIG. 11A depicts a portion of anvil 44 according to another embodiment of the invention. Welding line 68 of anvil 44 includes at least one notch 200 that has a cross-sectional area 216 that is smaller than the cross-sectional area 218 of a corresponding elastic thread 18 when in its non-tensioned state. Notch 200 forms a pair of adjacent bonds 100, 101 between first and second web layers 12, 16 that anchor the elastic thread 18 within a passage 104 defined between web layers 12, 16 and the pair of adjacent bonds 100, 101 as shown in FIG. 11B. Depending on the geometry of notch 200, operational parameters of ultrasonic bonding apparatus 38, and material selection of web layers 12, 16 and elastic thread 18, the resulting pair of bonds 100, 101 may be discrete, separated bond sites, similar to those shown in FIG. 4A, or connected by virtue of fusion bonding between one or both of the web layers 12, 16 and the surface of the elastic thread 18, similar to that shown and described relative to FIG. 4B. While notch 200 is depicted with a notch geometry similar to that of FIG. 8, it is contemplated that notch 200 may have any of the alternative geometries described above with respect to FIGS. 3, 6, 7, 9, and 10.

In addition to the projections 202, 204 that form bonds 100, 101, welding line 68 of FIG. 11A includes projections 230, 232 with land surfaces 234, 236 that form corresponding bonds 238, 240 between first and second web layers 12, 16. Notch 242 is defined between projection 202 and projection 230; notch 244 is defined between projection 204 and 232. Unlike notch 200, notches 242, 244 have respective cross-sectional areas 246, 248 that are larger than the cross-sectional area 218 of the corresponding elastic thread 18A, 18B when in its non-tensioned state. As shown in FIG. 11B, notches 242, 244 define passages 250, 252 between first and second web layers 12, 16 and respective bond pairs 100/238 and 101/240 in resulting elastic composite structure 254 that are larger than the cross-sectional area 218 of the non-tensioned elastic threads 18A, 18B. Elastic threads 18A, 18B are free to expand to their non-tensioned state within passages 250, 252. Bond pairs 100/238 and 101/240 thus serve to define a channel that contains elastic threads 18A and 18B but does not anchor the elastic threads 18A, 18B in position relative to first and second web layers 12, 16.

In one non-limiting embodiment notches 200, 242, and 244 of anvil 44 are manufactured using a multi-step machining process that includes machining a pattern of similarly sized “anchoring” notches on the face 50 of the anvil at the desired location of each notch 200, 242, 244. In the illustrated example, the manufacturing process would include initially machining notches 200, 242, and 244 to all have the notch geometry or profile of notch 200, as indicated by dashed lines 254, 256. In a subsequent machining step, additional material is removed from select notch locations to define the final notch geometry of the larger, non-anchoring notches 242, 244.

FIGS. 11A and 11B are to be understood as depicting one exemplary and non-limiting configuration of anchoring notch 200 and non-anchoring notches 242, 244. It is to be understood that alternative embodiments may include any combination or pattern of anchoring and non-anchoring notches 200, 242, 244 based on design considerations of the end product. Thus, a given welding line 68 may include a repeating pattern of one or more alternating anchoring notches and one or more non-anchoring notches or only one type of notch. Specific regions containing only anchoring notches or only non-anchoring notches may also be defined between two or more sequential welding lines 68 on the face 50 of the anvil 44.

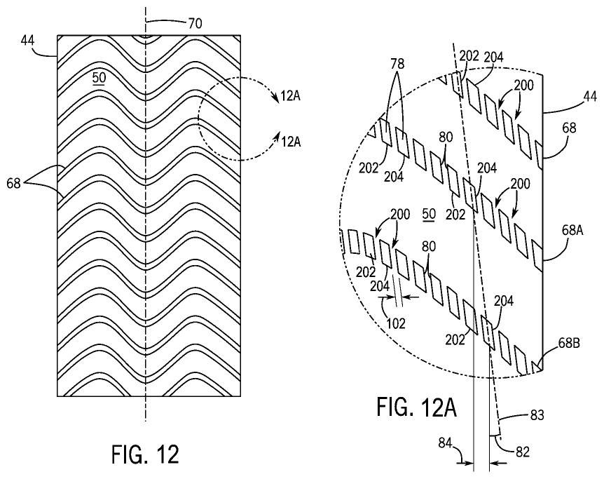

Referring now to FIG. 12, further details of the surface pattern of the anvil 44 is provided in accordance with one non-limiting embodiment of the invention. As shown, anvil 44 includes an array of welding lines 68 that are spaced apart from one another along the circumferential axis 70 of the anvil face 50. As shown more specifically in the detailed view provided in FIG. 12A, each welding line 68 contains a pattern of discrete projections 202, 204 that extend outward from the face 50 of the anvil 44. The projections 202, 204 are spaced apart from one another, by a gap that is defined by the width 102 of the notch 200 positioned between a given pair of adjacent projections 202, 204. Welding lines 68 are sinusoidal in the embodiment shown. However, may be straight lines, curved lines, or otherwise arranged to create a continuous and repeating pattern on the end product.

In the illustrated embodiment, the contact surfaces 78 of the projections 202, 204 have side surfaces 80 oriented at an angle 82 relative to the circumferential axis 70 such that no hypothetical arc 83 drawn from adjacent welding lines 68 is parallel to the circumferential axis 70 of the anvil 44. In such an embodiment, the facing surfaces 80 of adjacent projections 202, 204 are non-parallel to the circumferential axis 70 as shown. As a result, projections 202, 204 of adjacent welding lines 68 are not aligned with one another along the circumferential axis 70. Instead, a given projection 72A in one welding line 68A is offset from a given projection 72B in an adjacent welding line 68B by a pitch 84 defined by an angle 82. Projections 202, 204 thus define a threaded pattern that extends around the circumferential face 50 of the anvil 44.

It is contemplated that the contact surfaces 78 of the projections 202, 204 may have different geometries in alternative embodiments. As non-limiting examples, projections 202, 204 may be circular, rectangular, crescent shaped, or have irregular shapes that may be selected to form a desired overall pattern on the end product. In yet another embodiment, corresponding projections 202, 204 of adjacent welding lines 68A, 68B may be aligned with one another in a line parallel to the circumferential axis 70. Alternatively, projections 202, 204 of sequential welding lines 68A, 68B may be offset from one another in the cross-machine direction thereby defining a stepped or non-linear passage through the bond lines that are formed on the first and second web layers 12, 16.

FIG. 13 illustrates a portion of an elastic composite structure 86 output from the ultrasonic bonding apparatus 38. The elastic composite structure 86 is illustrated in an elongated state with elastic threads 18 stretched to a point where the first web layer 12 and second web layer 16 are substantially flat. As shown, the elastic composite structure 86 includes the first web layer 12, the second web layer 16, and a number of elastic threads 18 that are located between the first and second web layers 12, 16 and oriented along a longitudinal axis 88 of the elastic composite structure 86. While the illustrated embodiment includes three (3) elastic threads 18 it is contemplated that alternative embodiments may include a single elastic thread 18 or any number of multiple elastic threads 18 based on design specifications of the end product.

The ultrasonic bonding operation results in a continuous and repeating pattern of bond lines 90 that minor the welding lines 68 on the anvil 44 and bond or fuse the first web layer 12 to the second web layer 16. Thus, in embodiments where welding lines 68 are sinusoidal, the resulting bond lines 90 have a similar sinusoidal bond pattern. As shown in the detailed view provided in FIG. 13A, the tensioned elastic threads 18 extend along a passage 92 that is bounded by the gap 94 formed between the facing edges 96, 98 of a pair of adjacent bonds 100, 101 in each subsequent bond line 90. The gap 94 has a width defined by the width 102 of the notches 200 on the anvil 44. In the regions between the bond lines 90, the elastic threads 18 are free to swell or expand to their non-tensioned state. In their non-tensioned state, each elastic thread 18 has a cross-sectional area 218 that is smaller than the cross-sectional area of the passage 104 formed between each pair of adjacent bonds 100, 101 and the first and second web layers 12, 16. As a result, the elastic thread 18 is trapped or anchored between adjacent pairs of bonds 100, 101 and the first and second web layers 12, 16.

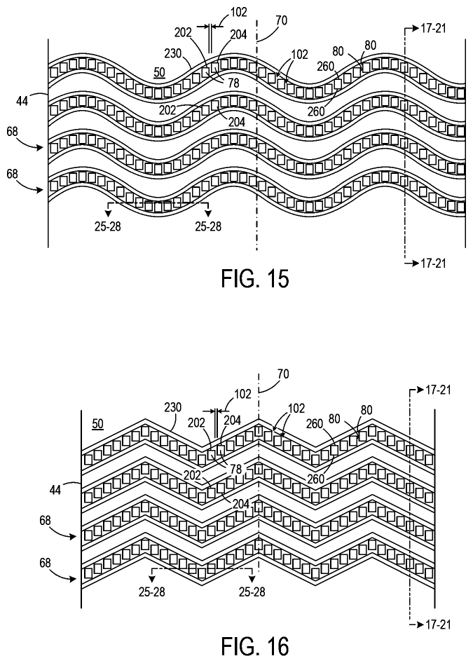

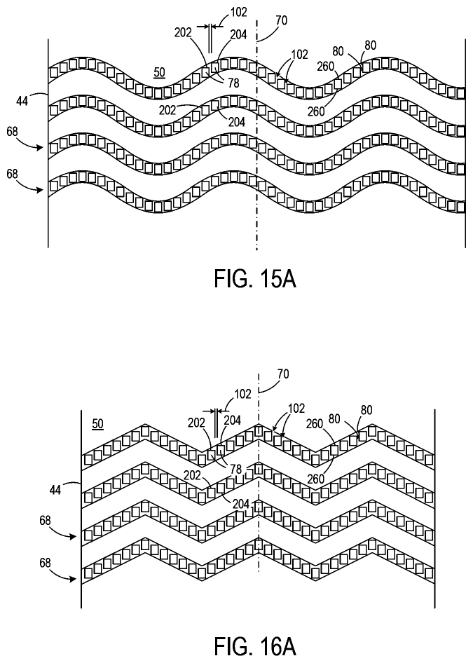

Referring now to FIG. 15, details of the surface pattern of the anvil 44 illustrated in FIG. 12 is provided in accordance with another non-limiting embodiment of the invention. As shown, anvil 44 includes an array of welding lines 68 that are spaced apart from one another along the circumferential axis 70 of the anvil face 50. Each welding line 68 contains a pattern of discrete projections 202, 204 that extend outward from the face 50 of the anvil 44. The projections 202, 204 are spaced apart from one another, by a gap that is defined by the width 102 of the notch 200 positioned between a given pair of adjacent projections 202, 204. Welding lines 68 are sinusoidal in the embodiment shown. In the embodiment shown in FIG. 16, the welding lines 68 follow a repeating chevron pattern. However, the welding lines 68 may be straight lines, curved lines, or otherwise arranged to create a continuous and repeating pattern on the end product.

Referring to FIGS. 15 and 16, in the illustrated embodiments, the contact surfaces 78 of the projections 202, 204 have side surfaces 80 aligned with the circumferential axis 70. Accordingly, the facing surfaces 80 of adjacent projections 202, 204 are parallel to the circumferential axis 70 as shown. End surfaces 260 of each projection 202, 204 are perpendicular to the facing surfaces 80 and are thus perpendicular to the circumferential axis 70. As a result, the end surfaces 260 of the projections 202, 204 in any of the welding lines 68 are parallel with one another no matter the position of any individual projection 202, 204 along the sinusoid or chevron pattern of the welding lines 68. The shape of at least the contact surfaces 78 of the projections is rectangular and may be square, for example. In addition, the shape of the entire projection may be a rectangular cuboid where any pair of opposite sides may be rectangular or square. The corners of the projections may be straight or radiused in alternative embodiments.

In an alternative embodiment, end surfaces 260 may be oriented at a common angle other than 90 degrees relative to the circumferential axis 70, with the end surfaces 260 of all projections 202, 204 in all welding lines 68 remaining parallel with one another regardless of the position of any individual projection 202, 204 along the repeating pattern of the welding lines 68. In such an embodiment, the shape of the contact surfaces 78 of the projections 202, 204 may be shaped as parallelograms.

It is contemplated that corresponding projections 202, 204 of adjacent welding lines 68 may be aligned with one another in a line parallel to the circumferential axis 70. Alternatively, projections 202, 204 of sequential welding lines 68 may be offset from one another in the cross-machine direction thereby defining a stepped or non-linear passage through the bond lines that are formed on the first and second web layers 12, 16. However, the alignment or misalignment of the adjacent welding lines 68 with respect to each other does not affect the arrangement of the end surfaces 260 of the projections 202, 204 being parallel to each other.

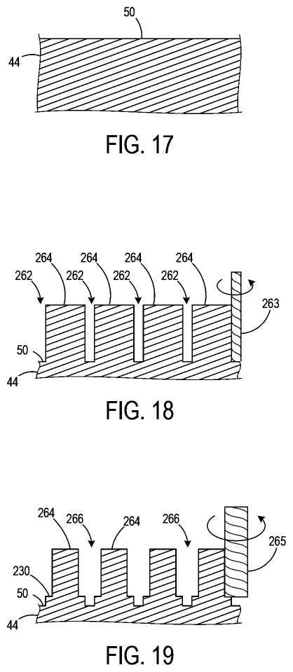

FIGS. 17-20 illustrate cross-sectional views taken along line 17-21 of FIGS. 15 and 16 showing steps for manufacturing the rotary anvils of FIGS. 15 and 16 according to an embodiment of the invention. FIG. 17 illustrates a portion of a blank rotary anvil 44, the face 50 of which is processed as described below.

As shown in FIG. 18, the face 50 of the anvil 44 is lowered as grooves 262 are formed through a removal process using, for example, a rotary milling or other cutting device 263. In another embodiment, a removal process such as electrical discharge machining as known in the art may be used. As a result of the removal process, a plurality of ridges 264 are created above the anvil face 50. The grooves 262 are spaced according to the desired positions of the welding lines 68. The formation of the grooves 262 helps shape the welding lines 68 into sinusoid, chevron, alternative non-linear patterns, or linear patterns as desired.

FIG. 19 illustrates an optional secondary material removal process that utilizes a second milling or cutting device 265 to remove additional bulk material from the grooves 262. In the illustrated embodiment, the second milling device 265 is wider than the cutting device 263 of FIG. 18; however, it can be sized differently in alternative embodiments. Milling device 265 creates larger grooves 266 that, when lined up with grooves 262 and when cutting to a shallower depth, create the steps 230 in the ridges 264. In alternative embodiments, cutting device 263 of FIG. 18 may be controlled to create the steps 230 in the ridges 254, the steps 230 may be made using an alternative material removal device such as an electrical discharge machining device, for example, or the steps 230 may be omitted entirely. In embodiments where the steps 230 are omitted, welding lines 68 may be formed having a non-linear sinusoidal or chevron pattern as shown in FIG. 15A and FIG. 16B, respectively, as non-limiting examples.

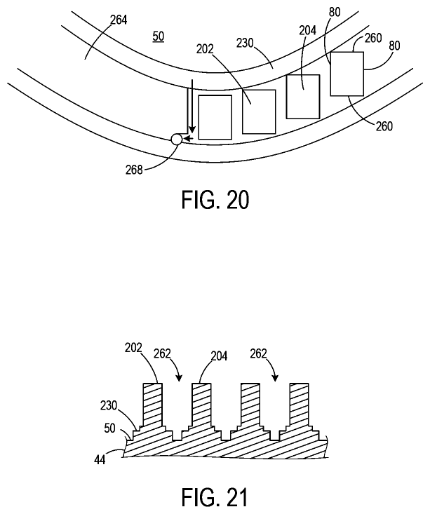

In a top view orientation, FIG. 20 illustrates the formation of individual protrusions 202, 204 in a ridge 264 of one of the weld lines 68. Another material removal device 268 such as a milling or cutting device may be used to remove material from the ridge 264 to create protrusions 202, 204 having parallel side surfaces 80 as well as parallel end surfaces 260 that are orthogonal to the side surfaces 80. In one embodiment, material removal device 268 may be an electrode of an electrical discharge machining (EDM) device used to remove material from the ridge 264 via electrical current discharging between the electrode and the workpiece (i.e., the ridge 264). The material removal device 268 may be configured to translate in a linear motion to remove material, as shown in FIG. 20, or via rotary motion. Removal of the material of the ridge 264 by the material removal device 268 forms a step 269 above the optional step 230 when included such that a pair of steps may exist between the anvil face 50 and the protrusions 202, 204.

A portion of a completed rotary anvil 44 is illustrated in FIG. 21 manufactured according to the steps shown in FIGS. 17-20.

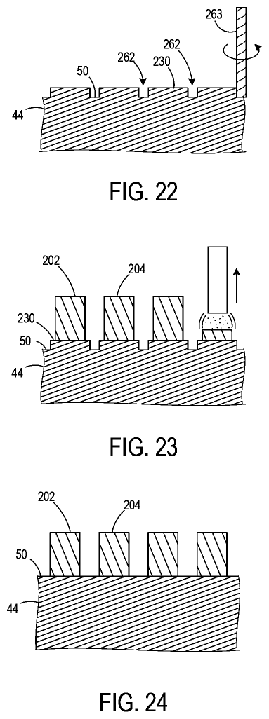

FIG. 24 illustrates a rotary anvil manufactured according to another embodiment of the invention. In this embodiment, the projections 202, 204 are built up from the face 50 of the rotary anvil 44 without creating the grooves 262 and steps 230 performed in the method illustrated in FIG. 18. Manufacture begins with the step shown in FIG. 22, wherein the face 50 of the anvil 44 provided in FIG. 17 is lowered as the grooves 262 are formed through a removal process using, for example, a rotary milling or other cutting device 263. Alternatively, material removal device 263 may be an electrode used in an electrical discharge machining device. Formation of the grooves 262 creates the steps 230 above the anvil face 50. The grooves 262 are spaced according to the desired positions of the welding lines 68. The formation of the grooves 262 helps shape the welding lines 68 into sinusoid or chevron patterns.

The manufacture of the projections 202, 204 occurs during a build-up or deposition process as illustrated in FIG. 23. The projections 202, 204 are grown from the surface of the individual steps 230 as material is deposited thereon. A controlled growing process creates the shapes of the contact surfaces 78 such that the facing surfaces 80 and the end surfaces 260 are aligned as described herein. In particular, the end surfaces 260 of each projection 202, 204 are parallel with each other no matter the position of the projection 202, 204 along the welding line pattern.

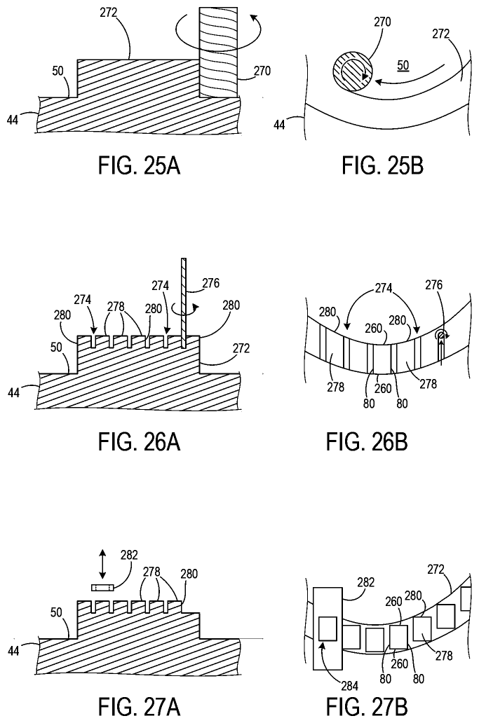

FIGS. 25A-28A illustrate cross-sectional views taken along line 25-28 of FIGS. 15 and 16 showing steps for manufacturing the rotary anvils of FIGS. 15 and 16 according to another embodiment of the invention. FIGS. 25B-28B illustrate top views corresponding to the steps shown in FIGS. 25A-28A.

As shown in FIGS. 25A and 25B, from the portion of blank rotary anvil 44 illustrated in FIG. 17, the face 50 of the anvil 44 is lowered through a removal process using, for example, a rotary milling or other cutting device 270. In another embodiment, a removal process such as electrical discharge machining as known in the art may be used. As a result of the removal process, a ridge 272 is created above the anvil face 50. The ridge 272 is shaped into sinusoid, chevron, alternative non-linear patterns, or linear patterns as desired.

FIGS. 26A and 26B illustrate a plurality of grooves 274 formed in the ridge 272 utilizing another milling or cutting device 276 to remove material from the ridge 272 to create land surfaces 278 of respective projections 280. In the illustrated embodiment, the milling device 276 is narrower than the cutting device 270 of FIG. 25A; however, it can be sized differently in alternative embodiments. Alternatively, cutting device 276 may be a rotary cutting device such as a circular saw having an axis of rotation orthogonal to the axis of rotation of the milling device 276 illustrated in FIGS. 26A, 26B. As illustrated, while the side surfaces 80 of the projections 280 are parallel to one another, the end surfaces 260 follow the path formed by the cutting device 270. Accordingly, the end surfaces 260 may not be parallel with each other and may not be perpendicular to the circumferential axis 70.

To shape the end surfaces 260 into a pattern where all end surfaces 260 of the projections 280 in any of the welding lines 68 are parallel with one another no matter the position of any individual projection 280 along the sinusoid, chevron, or other non-linear pattern of the welding lines 68, an electrode 282 of an electrical discharge machining device may be used as illustrated in FIGS. 27A, 27B to remove material from the projections 280 via electrical current discharging between the electrode 282 and the workpiece (i.e., the ridge 272). The electrode 282 has an opening 284 formed therein that corresponds with the desired finished shape of the land surface 278. As illustrated, a rectangular shape to the opening 284 creates rectangular land surfaces 278 where the end surfaces 260 of all projections 280 are parallel to each other and are perpendicular to the side surfaces 80 and to the circumferential axis 70.

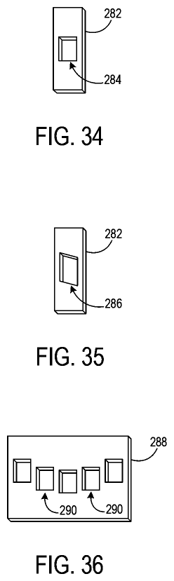

Referring now to FIG. 34, an orthogonal view of the electrode 282 is illustrated. The rectangular-shaped opening 284 corresponds to the shape of the land surfaces 278 illustrated in FIG. 25B. As shown in FIG. 35, other shapes of the opening 284 are contemplated herein such as the parallelogram-shaped opening 286, which created end surfaces 260 that are parallel to each other but which are not perpendicular to the side surfaces 80. FIG. 36 illustrates an electrode 288 with multiple openings 290 that can reduce manufacturing time by creating multiple projections 280 at the same time.

Referring back to FIGS. 27A and 27B, while the electrode 282 with the rectangular opening 284 is illustrated, the parallelogram opening 286 of the electrode 282 of FIG. 34 may be used instead, or an opening having a different shape than that illustrated in the figures herein may be used. For example, other shaped openings may be used that have circular, crescent shaped, or have irregular shapes that may be selected to form a desired overall pattern on the end product.

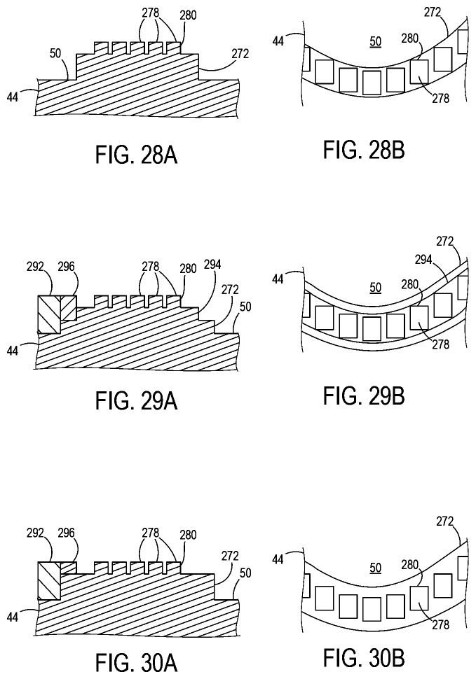

A portion of a completed rotary anvil 44 is illustrated in FIGS. 28A, 28B manufactured according to the steps shown in FIGS. 25A-28A.

FIGS. 29A, 29B illustrate a portion of a completed rotary anvil 44 manufactured according to another embodiment of the invention. The rotary anvil 44 of FIGS. 29A, 29B is manufactured similarly to the rotary anvil 44 of FIGS. 28A, 28B with the addition of an additional material removal step. As illustrated, in a bulk material removal step corresponding to a similar step illustrated in FIG. 26A, a portion 292 of the rotary anvil is removed that is smaller than the portion removed in FIG. 26A using cutting device 270. A step 294 may be created by removing an additional portion 296 of the anvil 44 such that less material is removed as compared with the rotary anvil 44 of FIGS. 28A, 28B. While portions 292, 296 are referenced on one side of the anvil 44, they are not shown on the other side of anvil 44 for clarity in illustrating the result of such removal. However, prior to the material removal, portions 292, 296 for material removal also correspond to the other side of the anvil 44.

As illustrated in FIGS. 30A, 30B, a portion of a completed rotary anvil 44 manufactured according to another embodiment of the invention. In the embodiment shown, portion 296 of anvil 44 refers to less material removed such that the ridge 272 is wider than that illustrated in FIGS. 28A, 28B. The use of a cutting device such as cutting device 276 to remove portion 296 reduces the amount of material to be removed by electrode 282.

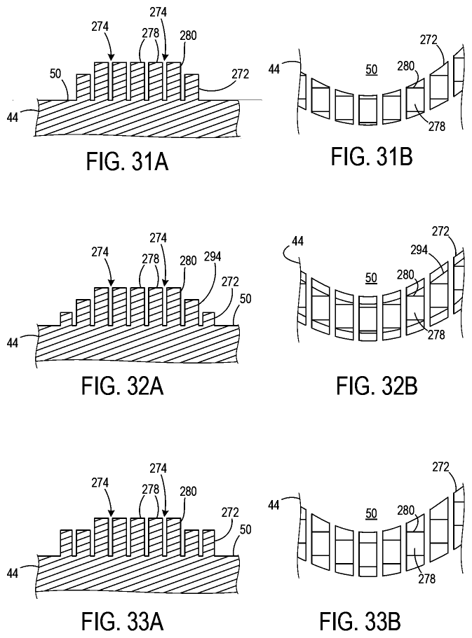

FIGS. 31A-33A and 31B-33B correspond with and are similar to the completed rotary anvils 44 illustrated in FIGS. 28A-30A and 28B-30B except for the depth of the grooves 274. As illustrated in FIGS. 31A-33A and 31B-33B, the depth of the grooves 274 extends to the depth of the anvil face 50. Accordingly, islands of individual protrusions 280, ridges 272, and optional steps 294 are formed.

The apparatus and methods described herein can be used to make elastic composite structures for waist regions, below-waist regions, and/or leg cuff regions of a single-piece or three-piece diaper, as non-limiting examples, without the use of glue. By eliminating the use of glue, the resulting elastic composite is softer to the touch and has a more uniform ruffling pattern in the cross-machine direction (i.e., the direction perpendicular to the machine direction). From a manufacturing standpoint, the elastic threads are anchored within dedicated passages of the elastic composite structure that are defined based on notch geometries of the bonding assembly that improve the reliability of the bonds that anchor the elastic threads in position and reducing the likelihood of thread breakage during manufacture. Accordingly, embodiments of the invention disclosed herein provide a more reliable manufacturing process than existing prior art approaches and result in an end product that is visually and tactilely more pleasing to the end customer.

Therefore, according to one embodiment of the invention, a rotary anvil comprises a face surface, a plurality of non-linear ridges defined by a first plurality of grooves in the face surface, and a plurality of projections in each of the plurality of non-linear ridges. Each projection of the plurality of projections comprises a contact surface having parallel facing surfaces and parallel end surfaces.

According to another embodiment of the invention, a method of manufacturing a rotary anvil comprises providing a rotary anvil having a face surface, removing material from the face surface to form a plurality of non-linear welding lines in the rotary anvil, and forming a plurality of projections in the plurality of non-linear welding lines. Forming the plurality of projections comprises creating a contact surface for each projection of the plurality of projections, the contact surface having parallel facing surfaces and parallel end surfaces. The parallel facing surfaces of each contact surface are parallel to one another, and the parallel end surfaces of each contact surface are parallel to one another.

According to yet another embodiment of the invention, an elastic composite structure comprises a first web layer, a second web layer coupled to the first web layer by a non-linear bond pattern comprising at least non-linear one bond line having at least one pair of adjacent bonds, and at least one elastic thread extending through a passage defined by facing edges of the at least one pair of adjacent bonds. Each bond in the at least one bond line comprises parallel facing surfaces and parallel end surfaces orthogonal to the parallel facing surfaces.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed is:

1. A rotary anvil comprising:
a face surface;
a plurality of non-linear ridges defined by a first plurality of grooves in the face surface;
a plurality of projections in each of the plurality of non-linear ridges; and
wherein each projection of the plurality of projections comprises a contact surface having parallel facing surfaces and parallel end surfaces.

2. The rotary anvil of claim 1, wherein the parallel facing surfaces are parallel to a circumferential axis of the rotary anvil.

3. The rotary anvil of claim 1, further comprising one or more steps within each of the plurality of ridges defined by a second plurality of grooves.

4. The rotary anvil of claim 3, wherein each projection of the plurality of projections extends from the one or more steps formed in a respective ridge of the plurality of ridges.

5. The rotary anvil of claim 1, wherein the parallel facing surfaces are parallel with a circumferential axis of the face surface.

6. The rotary anvil of claim 1, wherein the parallel facing surfaces are orthogonal to the parallel end surfaces.

7. The rotary anvil of claim 1, wherein the plurality of non-linear ridges are sinusoidal.

8. The rotary anvil of claim 1, wherein the plurality of non-linear ridges define a repeating chevron pattern.

9. The rotary anvil of claim 7, wherein each groove of a plurality of grooves in the plurality of ridges separates the parallel facing surface of one projection of the plurality of projections from the parallel facing surface of an adjacent projection of the plurality of projections.

10. A method of manufacturing a rotary anvil comprising:
providing a rotary anvil having a face surface;
removing material from the face surface to form a plurality of non-linear welding lines in the rotary anvil;
forming a plurality of projections in the plurality of non-linear welding lines;
wherein forming the plurality of projections comprises creating a contact surface for each projection of the plurality of projections, the contact surface having parallel facing surfaces and parallel end surfaces;
wherein the parallel facing surfaces of each contact surface are parallel to one another; and
wherein the parallel end surfaces of each contact surface are parallel to one another.

11. The method of claim 10 further comprising removing material from the plurality of non-linear welding lines to form a plurality of steps in the plurality of non-linear welding lines.

12. The method of claim 10, wherein the parallel facing surfaces are orthogonal to the parallel end surfaces.

13. The method of claim 10, wherein forming the plurality of grooves comprises forming the plurality of grooves via one of milling, cutting, and electrical discharge machining.

14. The method of claim 10, wherein the plurality of non-linear welding lines have one of a sinusoidal pattern and a repeating chevron pattern.

15. An elastic composite structure comprising:
a first web layer;
a second web layer coupled to the first web layer by a non-linear bond pattern comprising at least one non-linear one bond line having at least one pair of adjacent bonds; and
at least one elastic thread extending through a passage defined by facing edges of the at least one pair of adjacent bonds;
wherein each bond in the at least one non-linear bond line comprises:
parallel facing surfaces; and
parallel end surfaces orthogonal to the parallel facing surfaces.

16. The elastic composite structure of claim 15, wherein the non-linear bond pattern comprises a sinusoidal bond pattern.

17. The elastic composite structure of claim 15, wherein the non-linear bond pattern comprises a repeating chevron bond pattern.

18. The elastic composite structure of claim 15, wherein the non-linear bond pattern comprises a plurality of non-linear bond lines.

19. The elastic composite structure of claim 18, wherein the plurality of non-linear bond lines comprises a continuous and repeating pattern.

20. The elastic composite structure of claim 15, wherein each bond in the at least one bond line comprises:
facing surfaces; and
end surfaces;
wherein the facing surfaces of all bonds are parallel to each other; and
wherein the end surfaces of all bonds are orthogonal to the facing surfaces.
